(19) United States
(12) Patent Application Publication
(10) Pub. No.: US 2018/0289547 A1
(43) Pub. Date: Oct. 11, 2018

(54) SKI GOGGLES WITH REPLACEABLE NOSE BRIDGE SUPPORT

(71) Applicant: SHENZHEN PENGYIFA PRECISION MOULD CO., LTD, Shenzhen (CN)

(72) Inventor: Jianfeng WU, Shenzhen (CN)

(21) Appl. No.: 15/482,089

(22) Filed: Apr. 7, 2017

(51) Int. Cl. A61F 9/02

(52) U.S. Cl. CPC A61F 9/025; A61F 9/026

(57) ABSTRACT

The invention discloses a pair of ski goggles with a replaceable nose bridge support. The ski goggles comprise a ski goggle body and a sponge goggle frame and are characterized by further comprising a nose bridge support; the nose bridge support is located between the ski goggle body and the sponge goggle frame, the shape of the nose bridge support is identical with that of the ski goggle body and the sponge goggle frame, and a detachable structure is provided between the nose bridge support and the ski goggle body. Compared with the prior art, the invention discloses a pair of ski goggles with a replaceable bridge support, which has different specifications of nose bridge supports and sponge goggle frames for selection and replacement according to different face shapes and nose bridge heights and can be applicable to various persons of different face shapes and nose bridge heights.

### BACKGROUND

### 1. Technical Field

The invention relates to a pair of ski goggles, in particular to a pair of ski goggles with a replaceable nose bridge support.

### 2. Description of Related Art

A pair of common ski goggles is composed of a ski goggle body and a sponge goggle frame, but because the ski goggle body and the sponge goggle frame are integrally formed by adopting a fixed mold, and the nose bridge portion is not applicable to all persons of different face shapes and nose bridge heights, it is prone to cause that air easily enters the ski goggle body from a gap between the nose and the sponge goggle frame after the ski goggles are worn, such that not only cold air easily enters, but also it is also possible to cause fogging inside the ski goggles to affect the effects of the ski goggles, and it is prone to bring potential safety hazards to a person who is skiing.

### SUMMARY

Aiming at the shortcomings above in existing technology, the invention is to provide a pair of ski goggles with a replaceable nose bridge support, which has different specifications of nose bridge supports and sponge goggle frames for selection and replacement according to different face shapes and nose bridge heights and can be applicable to various persons of different face shapes and nose bridge heights.

For achieving the objective above, the solution is to provide a pair of ski goggles with a replaceable nose bridge support. The ski goggles comprise a ski goggle body and a sponge goggle frame. As a technical improvement of the present invention, the ski goggles further comprise a nose bridge support; the nose bridge support is located between the ski goggle body and the sponge goggle frame, the shape of the nose bridge support is identical with that of the ski goggle body and the sponge goggle frame, and a detachable structure is provided between the nose bridge support and the ski goggle body.

Preferably, magnet clamping grooves and magnet blocks are provided in corresponding positions of the nose bridge support and the ski goggle body respectively, and the magnet clamping grooves and the magnet blocks are uniformly distributed on frames of the nose bridge support and the ski goggle body.

Preferably, the magnet blocks are located inside the magnet clamping grooves, and the number of the magnet blocks is the same as that of the magnet clamping grooves.

Preferably, a pressing shifter is provided at each of the left side and the right side of the nose bridge support, and the pressing shifters are used for separating the nose bridge support from the ski goggle body,

Preferably, the nose bridge support is fixedly connected with the sponge goggle frame.

Preferably, the nose bridge support is provided with a first nose protrusion surface, the sponge goggle frame is provided with a second nose protrusion surface, and the radian of the first nose protrusion surface is identical with the shape of the second nose protrusion surface.

Preferably, assembling columns are provided on the nose bridge support, assembling holes are provided in positions, corresponding to the assembling columns, on the ski goggle body, the assembling columns are column bodies with column tips provided with short hooks, and each assembling hole is provided with a clamping position therein.

Preferably, connecting surfaces of the nose bridge support and the ski goggle body are integrally provided with a magic tape respectively.

Compared with the prior art, a pair of ski goggles with a replaceable nose bridge support disclosed by the present invention is a pair of ski goggles with a replaceable bridge support, which has different specifications of nose bridge supports and sponge goggle frames for selection and replacement according to different face shapes and nose bridge heights and can be applicable to various persons of different face shapes and nose bridge heights.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is an exploded drawing of the integral structure of the ski goggles with a replaceable nose bridge support of the invention;

FIG. 2 is a structure drawing of the integral structure of the ski goggles with a replaceable nose bridge support of the invention;

FIG. 3 is a schematic drawing of the nose bridge support of the ski goggles with a replaceable nose bridge support of the invention.

FIG. 4 is the structure drawing of the other embodiment of the invention;

FIG. 5 is the structure drawing of another embodiment of the invention;

Reference number in drawings are as follows in sequence:

1 nose bridge support; 2 ski goggle body; 3 sponge goggle frame; 4 magnet block; 5 magnet clamping groove; 6 pressing shifter; 7 first nose protrusion surface; 8 second nose protrusion surface; 41 assembling columns; 42 magic tape hook surface; 51 assembling holes; 52 magic tape hair surface.

### DETAILED DESCRIPTION

The present invention will be further described in detail as below in conjunction with the drawings and the embodiments, in order to make the objective, the technical solution and the advantages of the present invention understand more clearly. It should be understood that the specific embodiments described here are merely used for explaining the present invention, rather than limiting the present invention.

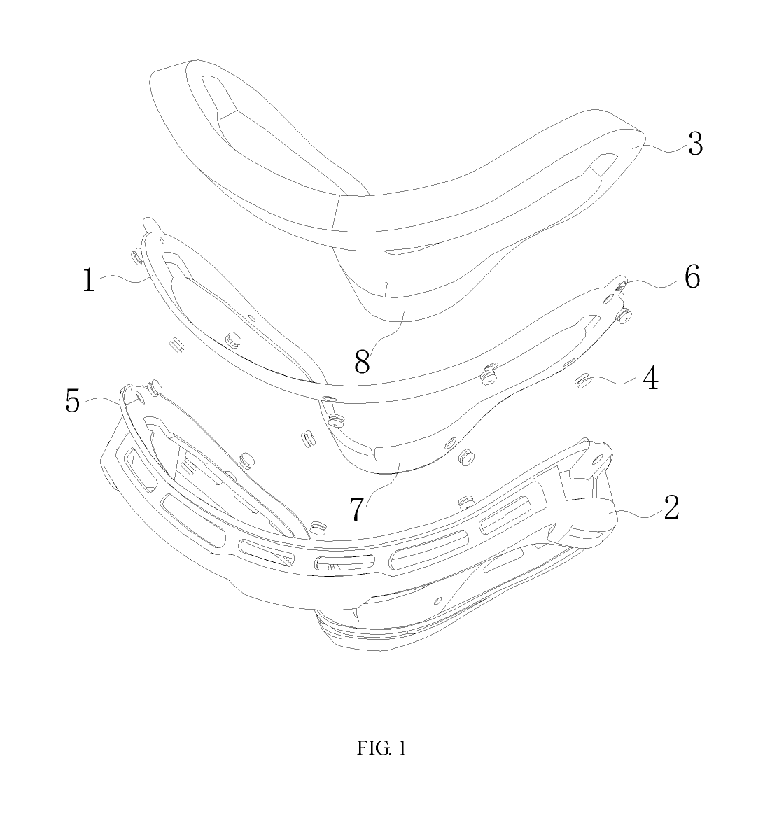

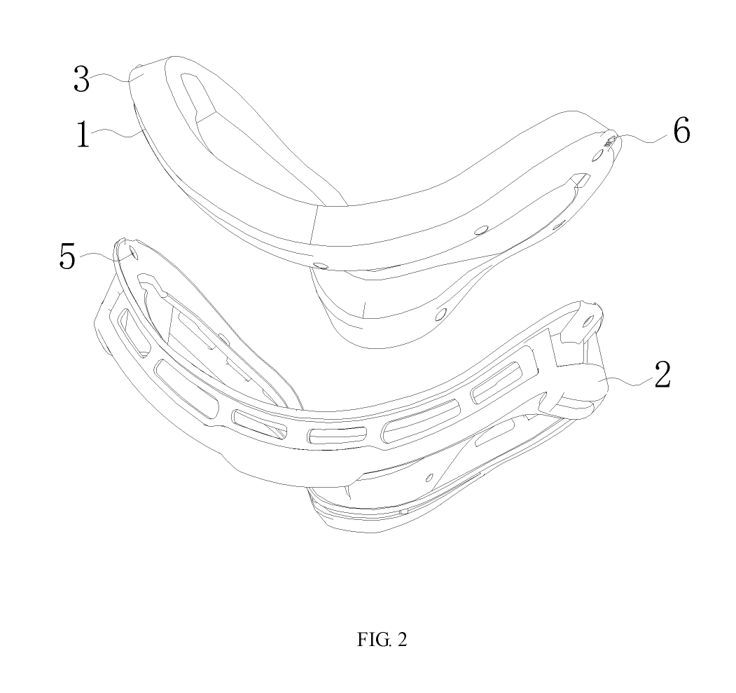

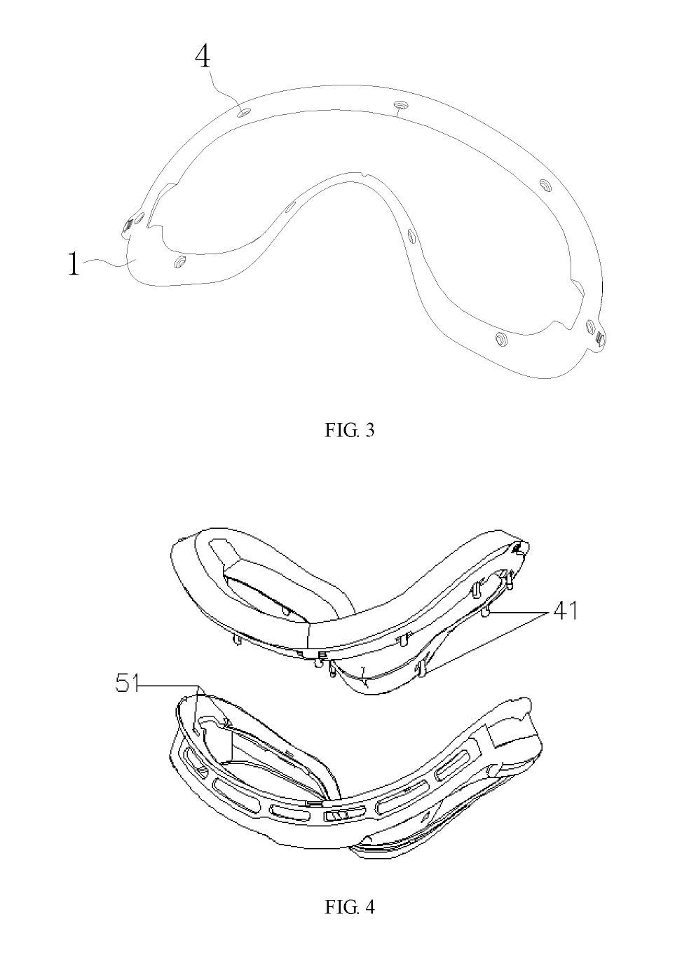

With reference to FIGS. 1-3, a pair of ski goggles with a replaceable nose bridge support disclosed by the present invention comprises a ski goggle body 2 and a sponge goggle frame 3. As a technical improvement of the present invention, the ski goggles further comprise a nose bridge support; the nose bridge support 1 is located between the ski goggle body 2 and the sponge goggle frame 3, the shape of the nose bridge support 1 is identical with that of the ski goggle body 2 and the sponge goggle frame 3, and a detachable structure is provided between the nose bridge support 1 and the ski goggle body 2.

A combination of the nose bridge 1 and the sponge goggle frame 3 can be replaced. There may be a plurality of assorted nose bridge supports 1 according to different nose shapes, and in practical use, the nose bridge support can be replaced according to the nose shape of the user to ensure a use effect of the ski goggles.

The nose bridge support 1 is magnetically connected with the ski goggle body 2. Magnet clamping grooves 5 and magnet blocks 4 are provided in corresponding positions of the nose bridge support 1 and the ski goggle body 2 respectively, and the magnet clamping grooves 5 and the magnet blocks 4 are uniformly distributed on frames of the nose bridge support 1 and the ski goggle body 2. The magnet blocks 4 are located inside the magnet clamping grooves 5, and the number of the magnet blocks 4 is the same as that of the magnet clamping grooves 5.

A pressing shifter 6 is provided at each of the left side and the right side of the nose bridge support 1, and the pressing shifters 6 are used for separating the nose bridge support 1 from the ski goggle body 2. Because the combination of the nose bridge support 1 and the sponge goggle frame 3 has elasticity, the nose bridge support 1 can be separated from the ski goggle body 2 just by pressing the pressing shifter 6.

The nose bridge support 1 is fixedly connected with the sponge goggle frame 3 in an adhering manner or other manners. The nose bridge support 1 is provided with a first nose protrusion surface 7, the sponge goggle frame 3 is provided with a second nose protrusion surface 8, and the radian of the first nose protrusion surface 7 is identical with the shape of the second nose protrusion surface 8. Replacements of the nose bridge support 1 and the sponge goggle frame 3 differ from originals thereof in that: the height of the first nose protrusion surface 7 is different from that of the second nose protrusion surface 8.

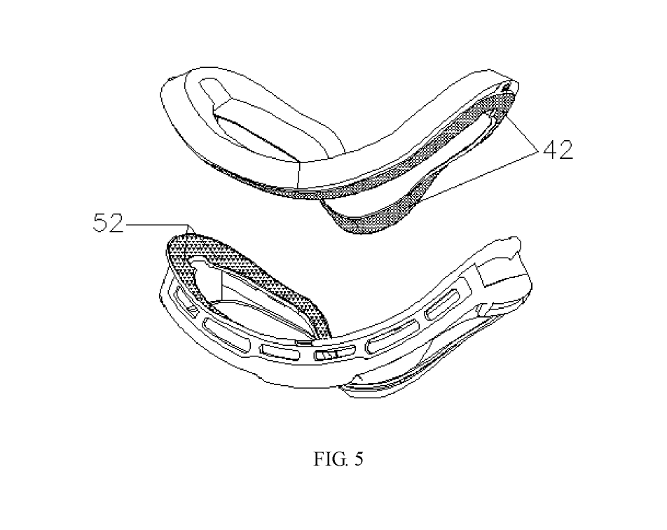

With reference to FIG. 4, in another embodiment of the present invention, assembling columns 41 are provided on the nose bridge support 1, assembling holes 51 are provided in positions, corresponding to the assembling columns 41, on the ski goggle body, the assembling columns 41 are column bodies with column tips provided with short hooks, and each assembling hole 51 is provided with a clamping position therein.

With reference to FIG. 5, in another embodiment of the present invention, connecting surfaces of the nose bridge support 1 and the ski goggle body 2 are integrally provided with a magic tape hair surface 52 and a magic tape hook surface 42 respectively, and the positions of the magic tape hair surface 52 and the magic tape hook surface 42 can be exchanged between the nose bridge support 1 and the ski goggle body 2.

In the implementation process, any one of magnet blocks, assembling columns or magic tapes can be provided between the ski goggle body 2 and the nose bridge support 1, but are not limited to detachable structures of other type.

Compared with the prior art, a pair of ski goggles with a replaceable nose bridge support disclosed by the present invention is a pair of ski goggles with a replaceable bridge support, which has different specifications of nose bridge supports and sponge goggle frames for selection and replacement according to different face shapes and nose bridge heights and can be applicable to various persons of different face shapes and nose bridge heights.

The foregoing descriptions of the embodiments and their accompanying drawings of the invention are intended to illustrate and not to limit this invention. Various changes and modifications may be made to the embodiments without departing from the spirit of the invention. Therefore, the scope of the invention is to be limited only by the appended claims.

What is claimed is:

1. A pair of ski goggles with a replaceable nose bridge support, comprising a ski goggle body and a sponge goggle frame, and further comprising a nose bridge support, wherein the nose bridge support is located between the ski goggle body and the sponge goggle frame, the shape of the nose bridge support is identical with that of the ski goggle body and the sponge goggle frame, and a detachable structure is provided between the nose bridge support and the ski goggle body.

2. The ski goggles with a replaceable bridge support according to claim 1, wherein magnet clamping grooves and magnet blocks are provided in corresponding positions of the nose bridge support and the ski goggle body respectively, and the magnet clamping grooves and the magnet blocks are uniformly distributed on frames of the nose bridge support and the ski goggle body.

3. The ski goggles with a replaceable bridge support according to claim 2, wherein the magnet blocks are located inside the magnet clamping grooves, and the number of the magnet blocks is the same as that of the magnet clamping grooves.

4. The ski goggles with a replaceable bridge support according to claim 1, wherein a pressing shifter is provided at each of the left side and the right side of the nose bridge support, and the pressing shifters are used for separating the nose bridge support from the ski goggle body.

5. The ski goggles with a replaceable bridge support according to claim 1, wherein the nose bridge support is fixedly connected with the sponge goggle frame.

6. The ski goggles with a replaceable nose bridge support according to claim 5, wherein the nose bridge support is provided with a first nose protrusion surface, the sponge goggle frame is provided with a second nose protrusion surface, and the radian of the first nose protrusion surface is identical with the shape of the second nose protrusion surface.

7. The ski goggles with a replaceable nose bridge support according to claim 1, wherein assembling columns are provided on the nose bridge support, assembling holes are provided in positions, corresponding to the assembling columns, on the ski goggle body, the assembling columns are column bodies with column tips provided with short hooks, and each assembling hole is provided with a clamping position therein.

8. The ski goggles with a replaceable bridge support according to claim 1, wherein connecting surfaces of the nose bridge support and the ski goggle body are integrally provided with a magic tape respectively.
